(19) United States
(12) Patent Application Publication
(10) Pub. No.: US 2007/0060937 A1
(43) Pub. Date: Mar. 15, 2007

(54) Safety needle holder

(76) Inventor: Chen Hai Liu, Taipei City (TW)

Correspondence Address: LEONG C LEI, PMB # 1008, 1867 YGNACIO VALLEY ROAD, WALNUT CREEK, CA, 94598, US

(21) Appl. No.: 11/213,758

(22) Filed: Aug. 30, 2005

(51) Int. Cl. A61B 17/34

(52) U.S. Cl. 606/185

(57) ABSTRACT

A safety needle holder for eye or body tattooing purpose that provides reliable operation of the needle, prevents the needle from falling out of its place, and avoids backflow of colorant is comprised of an upper holder containing a transmission shaft, a lower holder containing a needle, and a central holder connecting both of the upper and the lower holders; two flat sections being each made at where approaching both ends of the needle; two resilient members being each inserted to where above and beneath both flat sections; a sleeve being inserted to the lower end of the lower holder; and the needle being exposed out of the sleeve.

### BACKGROUND OF THE INVENTION

(a) Technical Field of the Invention

The present invention is related to a safety needle holder, and more particularly to one that permits easy, safe and reliable operation of the needle.

(b) Description of the Prior Art

The construction of a needle holder 1 usually operating for eyebrow and body tattooing of the prior art as illustrated in FIG. 1 of the accompany drawings is comprised of an upper holder 11 and a lower holder; and a transmission shaft 13 disposed in the upper holder 11 permits a needle 14 to penetrate through the bottom of the lower holder 12 and fixed to the transmission shaft 13. When replacement of the needle 14 is desired, the needle 14 is forthwith drawn out of the lower holder 12 to be also forthwith inserted with a new one. Therefore, the prior art is found with the following flaws:
1. If the needle 14 enters too deep into the lower holder, the transmission shaft could be easily deformed to affect the smooth operation of the needle.
2. On the contract, if the needle 14 enters too shallow, its engagement with the transmission shaft is incompletely to result in falling off (generally known as a flying needle) when the needle is working, which is a very dangerous situation.
3. The contact between the needle and the transmission shaft is poor and is vulnerable to backflow of the colorant.
4. Poor positioning of the needle 14 makes its unbalanced to easily swing and shake.

Furthermore, it is not convenient and it is noncompliant with hygienic requirements in the replacement of the needle.

### SUMMARY OF THE INVENTION

The primary purpose of the present invention is to provide a safe needle holder to permit more secured and reliable operation of the needle holder, allow easier replacement and operation, and prevent the needle from falling off its position for safety reasons.

To achieve the purpose, the safety needle holder of the present invention is comprised of an upper holder, a central holder, and a lower holder with a transmission shaft disposed in the upper holder and a needle disposed in the lower holder. Wherein, where approaching both ends of the needle, a flat section is each provided to the needle to be respectively inserted with a resilient member; and a sleeve is inserted to the lower opening of the lower holder for the needle to be exposed through the sleeve. Accordingly when the transmission shaft longitudinally drives the needle, the needle is well balanced and maintains its steadiness since the resiliency from the resilient member is greater than the travel spacing of the transmission shaft.

A tapered retainer is disposed to the lower edge of the lower holder and two pieces of foam plastic is respectively provided to where above and below the retainer as buffers to prevent the needle from shaking or swinging.

A through hole is disposed at the center of the sleeve while a recessed groove is provided at the bottom of the sleeve; and a tube protrudes from the center of the groove for a taper having provided at its center to be inserted into the groove for further improvement of the safety of the needle holder.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a sectional view of a needle holder of the prior art.

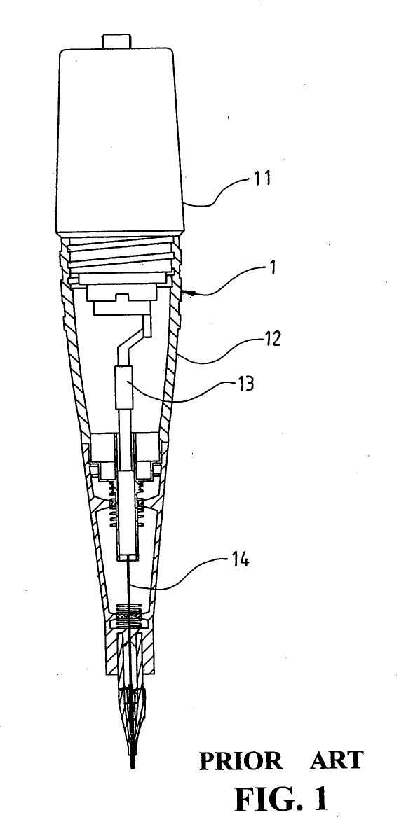

FIG. 2 is a perspective view of the present invention.

FIG. 3 is an exploded view of the present invention.

FIG. 4 is a sectional view of the present invention.

FIGS. 5A and 5B is a perspective view of a needle used in the present invention.

FIGS. 6A and 6B is a schematic view showing the replacement of a needle of the present invention.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

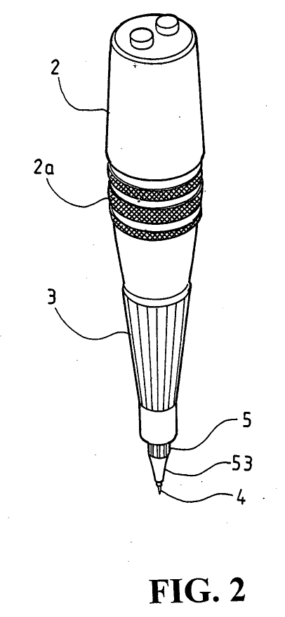

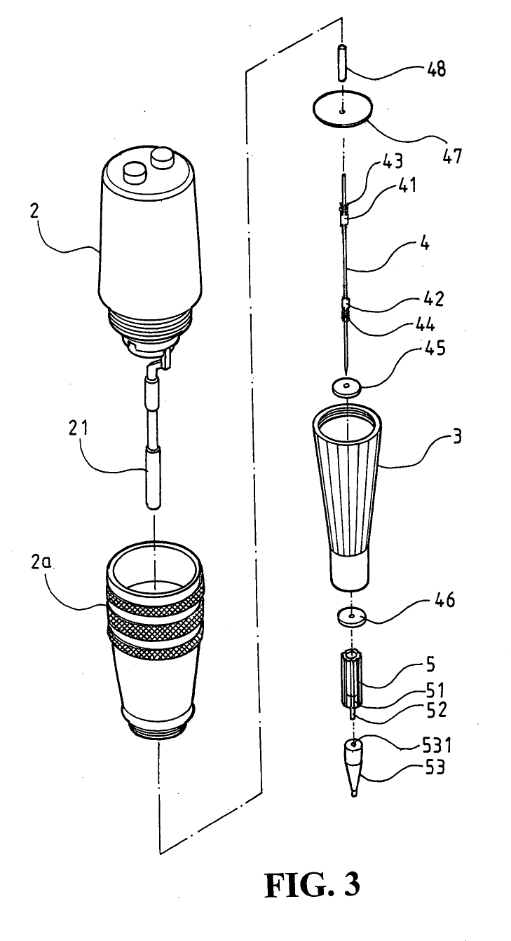

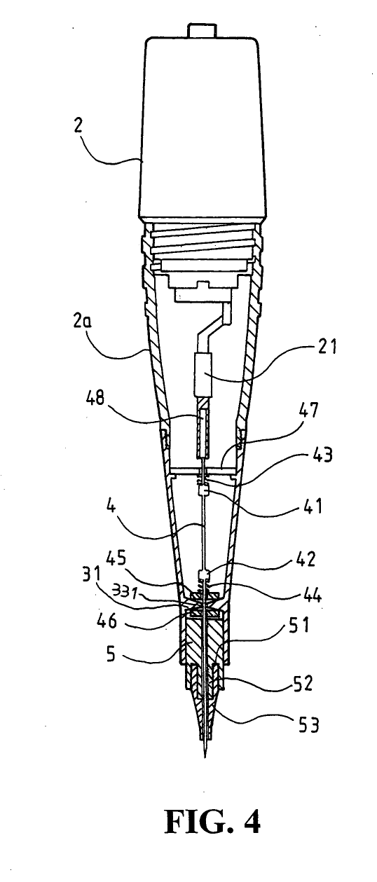

Referring to FIGS. 2, 3, and 4, a preferred embodiment of the present invention is essentially comprised of an upper holder 2, a central holder 2a, and a lower holder 3. Wherein, a transmission shaft 21 is provided to the upper holder 2, a retainer 31 with its upper and lower edges tapered is disposed at the lower end of the lower holder 3, a through hole 311 is disposed at the center of the retainer 31 to receive the insertion of a needle 4 in the lower holder 3.

Two flat sections 41, 42 are respectively provided to the needle 4 at where approaching both ends of the needle 4, and two resilient members 43, 44 are respectively inserted to where above the upper flat section 41 and where beneath the lower flat section 42; with the lower end of the needle 4 penetrating through the retainer 31 in the lower holder 3, two pieces of foam plastic 45, 46 are respectively inserted to the upper and the lower edges of the retainer; a lid 47 is inserted to the upper end of the needle 4 for the needle to be secured into an insertion piece 48.

A sleeve 5 is provided to an opening at the lower end of the lower holder 3, a recessed groove 51 is provided at the lower edge of the sleeve 5, and a tube 52 protruding from the center of the sleeve 5; and a taper 53 disposed at its center an insertion hole 531 is inserted into the groove 51 of the sleeve 5.

Where assembled, the needle 4 has its lower end to penetrate through the retainer 31 in the lower holder 3, then through the sleeve 5 to be exposed our of the sleeve 5 for a proper length. The upper end of the needle passes through the lid 47 and is secured in the insertion piece 48. The insertion piece 48 is secured to the transmission shaft 21 in the upper holder 2 to form a safety needle holder adapted to a tattoo machine for eye or body tattooing. In the use, the needle 4 as driven by the transmission shaft 21 engages longitudinal motion in cycle. Whereas the needle 4 is provided with two flat sections 41, 42 and two resilient members 43, 44, the resilience applied by both resilient members 43, 44 is greater than the travel spacing of the transmission shaft 21 when the needle 4 is active. Therefore, the needle 4 is kept firm and well balanced free of shaking or swinging. Furthermore, the needle 4 is kept more stable due to both pieces of foam plastic respectively disposed at where above and beneath the retainer 31 in the lower holder 3 serving as buffers while preventing backflow of the colorant. The needle holder is made safer since the active needle 4 is secured in its place to prevent potential danger of a flying needle.

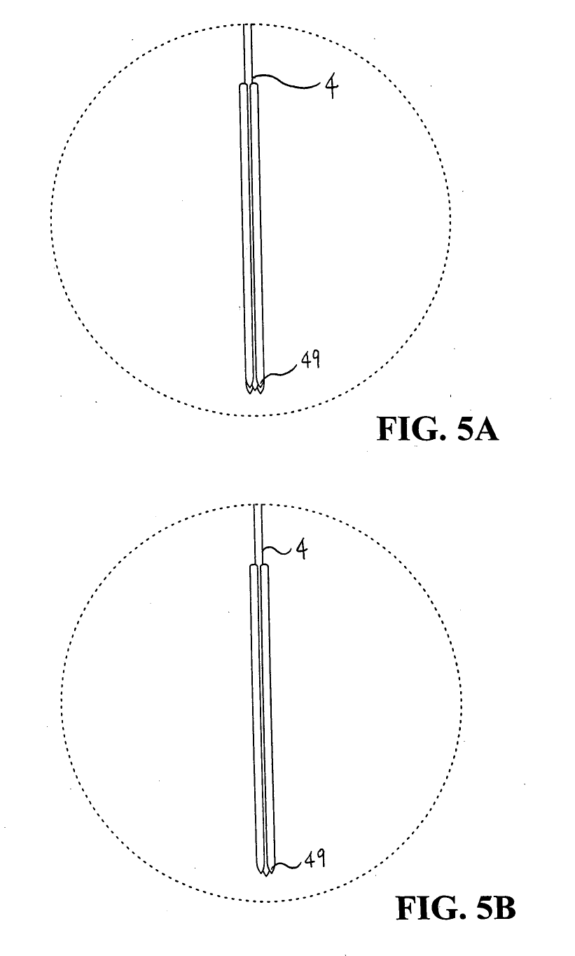

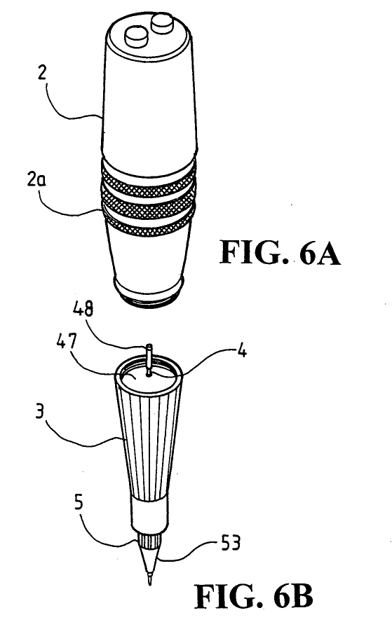

As illustrated in FIGS. 5A and 5B, multiple tips 49 may be arranged to the tail of the needle for providing more effects to the tattooing machine. Now referring to FIGS. 6A and 6B, the lower holder 3 is first removed by rotating it in case of replacing the needle 4, another lower holder 3 containing a needle of different size is fetched and engaged to the existing upper holder 2 without directly replacing the needle 4 to make the replacement of the needle safer and better compliance with hygienic requirements.

Accordingly, the present invention provides the following advantages including but not limited to:
1. The motion of the needle is free of the concerns of getting it too deep or shallow into the holder as found with the prior art; and the needle 4 is prevented from shaking or swinging due to that the resilience applied by both resilient members 43, 44 and both pieces of foam plastic 45, 46 is greater than the travel spacing of the transmission shaft 21.
2. The fear for the needle to fall out of its place while in use is eliminated since the insertion length of the needle is no longer an issue.
3. The internal mechanism of the tattooing machine will not be affected by the colorant thanks to the excellent tightness of the needle holder that effectively prevents backflow of colorant.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. A safety needle holder includes an upper holder containing a transmission shaft; a lower holder, having disposed at its button a retainer with tapered upper and lower edges, a through hole disposed at the center of the retainer, and a lid disposed on the upper end; a central holder connecting both of the upper and the lower holder a needle, having two flat sections each disposed at where approaching both ends of the needle, one resilient member being inserted to where above the upper flat section and another resilient member being inserted to where below the lower flat section, and an insertion piece being provided to the top of the needle; a sleeve inserted to an opening disposed at the bottom of the lower holder, having provided at its lower edge a recessed groove, a tube protruding from the center of the sleeve to receive insertion of a taper, and the tape being disposed at its center an insertion hole; the needle being disposed in the lower holder and penetrating through the lid and the retainer before being secured to the transmission shaft by means of the insertion piece; and the lower end of the needle being exposed out of the sleeve to form a safety needle holder to be driven by the transmission shaft for eyebrow or body tattooing purpose.

2. The safety needle of claim 1, wherein, two pieces of foam plastic are respectively disposed to the upper and the lower ends of the retainer as buffers to further secure the needle in operation.

3. The safety needle of claim 1, wherein the tail of the needle in the lower is made in the form of an arrangement of multiple tips to promote its practical use.
